(19) United States
(12) Patent Application Publication
(10) Pub. No.: US 2002/0147548 A1
(43) Pub. Date: Oct. 10, 2002

(54) Basecalling system and protocol

(76) Inventors: Dirk Walther, San Mateo, CA (US); Gabor T. Bartha, Mountain View, CA (US); MacDonald S. Morris, Felton, CA (US)

Correspondence Address: BEYER WEAVER & THOMAS LLP, P.O. BOX 778, BERKELEY, CA, 94704-0778, US

(21) Appl. No.: 09/927,321

(22) Filed: Aug. 10, 2001

Related U.S. Application Data
(60) Provisional application No. 60/225,083, filed on Aug. 14, 2000.
(60) Provisional application No. 60/257,621, filed on Dec. 20, 2000.

(51) Int. Cl. G06F 19/00; G01N 33/50

(52) U.S. Cl. 702/20

(57) ABSTRACT

Using data electrophoretic trace data from conventional nucleic acid sequencing equipment, a method for base calling that is tolerant to variable peak spacing is described. The method generates high-quality basecalls and reliable quality scores. In addition, a new type of quality score that estimates the probability of a deletion error between the current and the following basecall is described. A new protocol for benchmarking that better discerns basecaller performance is also provided.

### CROSS-REFERENCE TO RELATED PATENT APPLICATIONS

This application claims priority from U.S. Provisional Patent Application number 60/225,083, filed Aug. 14, 2000, by Walther et al., and titled BASE CALLING SYSTEM AND PROTOCOL; and U.S. Provisional Patent Application number 60/257,621, filed Dec. 20, 2000 by Walther et al., and titled BASE CALLING SYSTEM AND PROTOCOL. Each of these applications is incorporated herein by reference for all purposes.

### COPYRIGHT NOTICE

A portion of the disclosure of this patent document contains material which is subject to copyright protection. The copyright owner has no objection to the xeroxographic reproduction by anyone of the patent document or the patent disclosure in exactly the form it appears in the Patent and Trademark Office patent file or records, but otherwise reserves all copyright rights whatsoever.

### SOFTWARE APPENDIX

This specification includes Software Appendix on CD ROM having 1 file named LifeTrace Basecaller.txt which is 21 kb in size, and is incorporated herein by reference.

### FIELD OF THE INVENTION

This invention relates to the field of bioinformatics. More specifically, the present invention relates to computer-based methods and systems and media for evaluating biological sequences.

### BACKGROUND OF THE INVENTION

DNA sequencing usually begins with a purified DNA template upon which a reaction is performed for each of the four nucleotides (bases) generating a population of fragments that have various sizes depending on where the bases occur in the sequence. The fragments are labeled with base-specific fluorescent dyes and then separated in slab-gel or capillary electrophoresis instruments. As the fragments migrate past the detection zone of the sequencer, lasers scan the signals. Information about the identity of the nucleotide bases is provided by a base-specific dye attached to the primer (dye-primer chemistry) or dideoxy chain-terminating nucleotide (dye-terminator chemistry). Additional steps include lane tracking and profiling (slab-gel only) and trace processing which produces a set of four arrays (traces) of signal intensities corresponding to each of the four bases over the many time points of the sequencing run. Trace processing consists of baseline subtraction, locating start and stop positions, spectral separation, resolution enhancement, and some mobility correction. The final step in DNA sequencing is translating the processed trace data obtained for the four different bases into the actual sequence of nucleotides, a process referred to as base calling.

Approaches to the base calling problem include neural networks (U.S. Pat. Nos. 5,365,455 & 5,502,773), graph theory, homomorphic deconvolution (Ives et al. (1994) IEEE Transactions on Biomedical Engineering 41:509 and U.S. Pat. No. 5,273,632), modular (“object oriented”) feature detection and evaluation, classification schemes (PCT Publication No. WO 96/36872), correlation analysis, and Fourier analysis followed by dynamic programming. Additional related patents describe base-calling by blind deconvolution combined with fuzzy logic (PCT Publication No. WO 98/11258), by comparison to a calibration set of two-base prototypes in high dimensional “configuration space” (PCT Publication No. WO 96/35810), and by comparison to singleton peak models (PCT Publication No. WO 98/00708).

The accuracy of the computational algorithm employed for base calling directly impacts the quality of the resulting sequence, determines to a significant degree the economic costs associated with sequencing, and its usability for detecting Single Nucleotide Polymorphisms (SNPs). While base calling is algorithmically straightforward for ideal data (noise-free, evenly spaced, Gaussian-shaped peaks of equal height for all four bases), it is naturally more difficult and error prone for real trace data. Inevitable experimental as well as systematic factors degrade the quality of obtainable data resulting in peaks with variable spacing and height, with secondary peaks underneath the primary peak etc. See, e.g., Ewing et al. (1998) Genome Res. 8: 175-185.

Since base calling is error prone it is desirable to provide for each assigned base an estimate of confidence (quality score). The estimation of confidence is an integral part of many existing base calling algorithms. See, e.g., Giddings (1993) Nucleic Acids Res. 21: 4530-4540; Golden et al. (1993) Proceedings of the first International Conference on Intelligent Systems for Molecular Biology (ed. Hunter, L., Searls, D., Shavlick, J.): pp136-144. AAAI Press, Menlo Park, Calif.; Giddings (1998) Genome Res. 8: 644-665; and Ewing et al. (1998) Genome Res. 8: 186-194. Quality scores are critical for accurate sequence assembly and reliable detection of Single Nucleotide Polymorphisms (SNPs). See, e.g., Buetow et al., (1999) Nat Genet. 21: 323-325; and Altshuler et al. (2000) Nature. 407: 513-516. The rigorous implementation of the concept of quality scores that translate directly into an estimated error rate along with highly reliable basecalls for slab-gel based sequencing machines, helped phred, to become the most widely used base calling software. See, Richterich (1998) Genome Res. 8:251-259.

Significant problems have been noted with phred's algorithm for handling variable peak spacing, especially for MegaBACE sequencers where the spacing between peaks can change rather abruptly along the traces (commonly referred to as the “accordion effect”). Phred starts the base calling process by predicting idealized peak locations, which are then matched up with observed peaks to generate the actual calls. The problems are due to the way that phred computes and uses predicted peak information. Phred first looks for the portion of the chromatogram that has the most uniform spacing and works its way outward. At each step of the way out there is a limit on how fast the spacing can change. When the spacing changes too rapidly, phred can lose synchronization with the actual spacing. Attempts to improve phred's ability to handle variable peak spacing were met with limited success. When desynchronization occurs, phred may add or remove basecalls to preserve uniform peak spacing. This can result in excessive insertion and deletion errors that can lead to serious assembly problems or frame shifts during translation into amino acid sequence.

Improved computer systems and methods are still needed to evaluate, analyze, and process the vast amount of information now used and made available from DNA sequencing efforts.

### SUMMARY OF THE INVENTION

Using multiple noisy peak-like signals that vary in space or time as input, the present invention determines the sequence of peaks (and thus, basecalls) through a process that combines resolution enhancement and peak detection. This method places a higher emphasis on peak detection and/or assignment and local peak spacing estimation than the prior art methods that rely upon the estimation of global peak spacing. Because of these attributes, the methods described herein is robust with regard to variable peak spacing.

More specifically, the method generates a new trace (referred to as LT) by combining the information contained in the input traces. The trace LT is computed by cross-correlating every trace position and its vicinity to an ideal, Gaussian-shaped model peak. The newly generated, transformed traces are then combined to yield the LT-trace. The initial cross-correlation step improves the detection of peak-like shapes and allows for a better resolution of peaks without the need to analyze all input traces independently.

In a preferred embodiment, the invention provides base calling software (referred to as “LifeTrace”) that implements a novel algorithm for base calling from sequencing chromatogram trace data. The base calling method described herein utilizes call quality scores (described below), local peak spacing estimation, and other quality thresholds for removing, merging, and adding basecalls.

Another embodiment of the invention provides a new quality score: the gap-quality score. The gap quality score estimates the probability that between the current and the next base might be another base; i.e. that a deletion error has occurred. This new quality score allows for the identification of real deletions (deletion Single Nucleotide Polymorphisms) that occur as natural variations between individuals.

LifeTrace also computes traditional quality scores for each basecall. Phred uses a lookup-table (i.e., discontinuous) approach to match trace parameters with quality scores/observed error rates. The present invention provides for improved computing call quality scores and methods for their determination wherein continuous parameters are used to judge call quality.

The present invention also provides a method of sequence alignment that incorporates call quality and gap-quality scores in the dynamic programming method. As described below, this method of sequence alignment is useful for benchmarking the performance of basecallers. In addition, it can be used to calibrate quality scores.

Another aspect of the invention provides a method for comparing the performance of base calling algorithms that better discerns the performance differences than prior methods. According to the method of this invention, error statistics are collected over an extended sequence. More specifically, the present invention analyzes a region of sequence whose boundaries are determined by the furthest most high quality alignments contributed by either of the algorithms being benchmarked. Preferably, this method of benchmarking uses the alignment method described herein.

These and other features and advantages of the present invention will be described below in more detail with reference to the associated drawings.

### BRIEF DESCRIPTION OF THE DRAWINGS

FIG. 1 is a process flow diagram depicting—at a high level—one process of the invention for base calling.

FIG. 2 illustrates the processing of chromatogram trace data by LifeTrace. Shown are the four original data traces and the composite trace LT that provides the basis for peak detection. LifeTrace basecalls are given in the top row with the length of the tick lines that indicate the peak location corresponding to the LifeTrace quality score with longer ticks indicating higher quality. The two horizontal lines mark quality score zero and 15.

FIG. 3 is a process flow diagram depicting—at a high level—one process of the invention for calculating quality scores.

FIG. 4 illustrates the concept of a gap-quality. Part of a sample chromatogram shows traces and calls with associated quality scores quantified by the length of the peak locator tick mark. Two horizontal lines mark quality score levels of zero and 15. The left tick line represents the quality score of the actual base call, while the right tick line measures the quality of the gap to the following called base.

FIG. 5 is a process flow diagram depicting—at a high level—one process of the invention for the performance of quality filtering on called bases.

FIG. 6 is a block diagram of a computer system that may be used to implement various aspects of this invention such as the various base calling algorithms of this invention.

FIGS. 7A and 7B show a performance comparison phred (gray bars) and LifeTrace (black bars) using Method 1 (see section Performance analysis). Basecall errors are analyzed for the different error types and as a function of position in the called sequence. Panel A MegaBACE dye-primer set, b) MegaBACE dye-terminator set. ‘InDel’ combines insertions and deletion errors. ‘N’ refers to called ‘N’s; i.e. undecided basecalls.

FIG. 8, Panel A is a sample MegaBACE chromatogram with corresponding basecalls. Top row basecalls generated by phred, bottom row was called by LifeTrace. Length of peak locator tick lines corresponds to associated quality scores with longer ticks indicating higher quality. Horizontal lines mark quality score levels of zero and 15, respectively. Panel B shows peak-peak distance as a function of peak location as determined by LifeTrace. For every peak at a given chromatogram location (x-value) its associated distance to the next peak is plotted (y-value). The chromatogram segment shown in Panel A corresponds to chromatogram location between 4000 and 4400.

FIG. 9 shows a comparison of LifeTrace error rate to phred error rate in subsets of chromatogram grouped according to quality of the chromatogram. Here, quality is expressed as the maximum allowed number of basecall errors made by either LifeTrace or phred; i.e. max(LifeTrace_errors, phred_errors). For example, chromatograms for which both LifeTrace and phred generate fewer than 5 basecall errors can be considered high quality chromatograms. As the graph is demonstrating, LifeTrace outperforms phred in a set of chromatograms for which phred generates many errors, but LifeTrace only makes very few. Error rates are normalized by the number of phred errors, i.e. phred is the horizontal line at relative error rate 1. Broken lines correspond to the cumulative sum of the number of chromatograms normalized by the total number of chromatograms in the set at a given error threshold with the color code matching the legend colors.

FIG. 10 depicts the fidelity of LifeTrace and phred quality scores. Quality scores associated with all basecalls aligned to the true sequence were binned into intervals of width Δ(q-score)=2. Semi-logarithmic plot shows observed error rate in each bin as a function of quality score associated with that bin for the dye-primer and dye-terminator MegaBACE chromatogram set analyzed. Only substitution and insertion errors are considered here as deletion errors are captured by the newly introduced gap-quality score (see FIG. 13), and a deleted base itself does not have a quality as it does not exist. ‘Ideal’ refers to the ideal line of q=−10×log₁₀ (observed error rate).

FIG. 11 shows the discriminative power of quality scores and retention of high-quality base calls. Frequency distribution of quality scores associated with substitution and insertion errors and all basecalls for basecallers LifeTrace and phred for the chromatogram sets examined. Frequencies are computed for calls binned into intervals of width 2 units of quality scores.

FIG. 12 illustrates the fidelity of LifeTrace gap-quality scores. Semi-logarithmic plot of observed frequency of deletion errors as a function of assigned gap-quality score of the preceding base in the alignment for the MegaBACE chromatogram sets (primer and terminator) analyzed. The gap-quality score of the base preceding the gap captures the quality of the gap to the next called base, i.e. low gap-qualities indicate a high probability that another base might be between this and the next called base indicating a high likelihood of a deletion error. In LifeTrace gaps are considered a call and ‘observed error rate’ refers to the fraction of incorrect gaps (missed true basecall in between) out of all called gaps. Bin width was 4 quality units and ‘ideal line’ is as in FIG. 10.

FIG. 13 depicts the discriminative power of LifeTrace gap-quality scores. Frequency distribution of quality scores associated with deletion errors (gap-quality assigned to the gap-preceding basecall) and all gap calls for basecaller LifeTrace for the chromatogram sets examined. Frequencies are computed for calls binned into intervals of width 2 units of quality scores.

### DETAILED DESCRIPTION OF THE INVENTION

Overview

Generally, this invention relates to base calling processes (methods) and apparatus configured for base calling. It also relates to machine-readable media on which is provided instructions, data structures, etc. for performing the processes of this invention. In accordance with this invention, signals from the electrophoretic separation of DNA are manipulated and analyzed in certain ways to extract relevant features. Using those features, the apparatus and processes of this invention, can automatically draw certain conclusions about the sequence of the DNA. More specifically, the invention provides high-quality basecalls and reliable quality scores. The invention also provides a new type of quality score associated with every basecall, the gap-quality, which estimates the probability of a deletion error between the current and the following basecall. A new protocol for benchmarking that better discerns basecaller performance differences than previously published methods is also described.

Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference. Nothing herein is to be construed as an admission that the invention is not entitled to antedate such disclosure by virtue of prior invention.

“Electrophoresis” refers to the separation of molecules by differential molecular migration in an electric field. For biopolymers, this is ordinarily performed in a polymeric gel, such as agarose or polyacrylamide, whereby separation of biopolymers with similar electric charge densities, such as DNA and RNA, ultimately is a function of molecular weight.

“Data trace” refers to the series of peaks and valleys representing the migrating bands of oligonucleotide fragments produced in one chain termination sequencing reaction and detected in a DNA sequencer. The data trace may be either a raw data trace or a “processed” data trace.

The invention will now be described in terms of particular specific embodiments as depicted in the drawings. However, as will be apparent to those skilled in the art, the present invention may be practiced without employing some of the specific details disclosed herein. Some operations or features may be dispensed with. And often alternate elements or processes may be substituted.

A. Apparatus and Method for Base calling

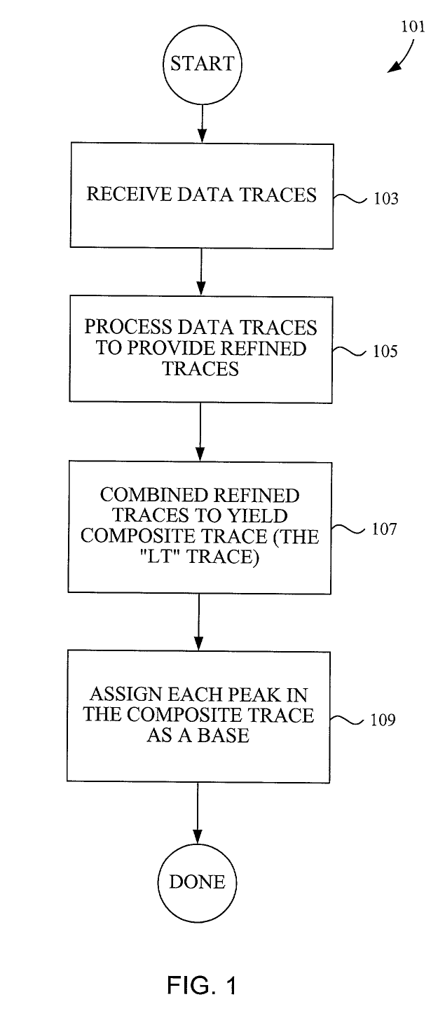

A high level process flow 101 in accordance with one embodiment of this invention is depicted in FIG. 1. As shown, the process begins at 103 where a sequence data processing tool receives data from an electrophoresis detection instrument. Such data is representative of the nucleic acid sequence of the sample material and, depending on the precise nature of the instrument, may have undergone some minimal level of processing (as discussed further below) before transmission. Alternatively, the sequence trace data processing tool can be integral to an electrophoresis detection instrument.

The data trace which is processed in accordance with the method of the invention is preferably a signal collected using the fluorescence detection apparatus of an automated DNA sequencer. However, the present invention is applicable to any data set which reflects the separation of oligonucleotide fragments in space or time, including real-time fragment patterns using any kind of detector, for example a polarization detector as described in U.S. Pat. No. 5,543,018; densitometer traces of autoradiographs or stained gels; traces from laser-scanned gels; and fragment patterns from samples separated by mass spectroscopy.

The electrophoresis detection instrument or DNA sequencer may utilize a variety of electrophoretic means to separate DNA, including without limitation, slab gel electrophoresis, tube gel electrophoresis, or capillary gel electrophoresis. Existing automated DNA sequencers are available from Applied Biosystems, Inc. (Foster City, Calif.); Pharmacia Biotech, Inc. (Piscataway, N.J.); Li-Cor, Inc. (Lincoln, Nebr.); Molecular Dynamics Inc. (Sunnyvale, Calif.); and Visible Genetics, Inc. (Tortonto). The methods described herein can be used with any of a variety of sequencing machines, including without limitation, the MegaBASE 1000 capillary sequencer available from Amersham; the ABI-3700 capillary sequencer, available from Applied Biosystems; and the ABI-377 slab gel sequencing machine, available from Applied Biosystems.

As described above, preferably, the data traces will be processed prior to analysis using the base calling methods described herein. More specifically, the electrophoretic data will undergo trace processing. Such trace processing methods are well known in the art and may consist of baseline subtraction, locating start and stop positions, spectral separation, resolution enhancement, and some mobility correction.

Occasionally, trace values exceed the upper detection threshold of the instrument and are clipped beyond this value, producing flat peaks. As such, the pre-processing step optionally may include the replacement of clipped peaks by caps conforming to a quadratic function, thus, rendering the clipped peak more peak-like. Alternatively, this may occur as part of the LifeTrace algorithm described herein.

More specifically, it is useful to locate the so-called “primer peak” and “termination peak” (i.e., the begin and end points) which are found in some variations of the chain-termination sequencing method. These peaks comprise a large volume of unreacted primer, which tends to interfere with base calling around the shorter chain extension products, and a large volume of the complete sequence which may interfere with base calling around the longest chain-extension products. These peaks are identified and eliminated from consideration either on the basis of their size, their location relative to the start and end of the electrophoresis process, or some other method.

After elimination of the primer and termination peaks, the data trace may be normalized so that all of the identified peak have the same the same height which is assigned a common value. This process reduces signal variations due to chemistry and enzyme function, and works effectively for homozygous samples and for many heterozygotes having moderate, i.e., less than about 5 to 10%, heterozygosity in a 200 base pair or larger region being sequenced. Spectral separation, spectral deconvolution or multicomponent analysis refers to the process of decorrelation of the raw fluorescence signal into the components produced by individual dyes, each dye representing one “color”. Color separation may be accomplished by least squares estimating wherein the raw data is fit to the dye spectra., e.g., singular value decomposition (SVD), or using other methods known in the art

Dye mobility shifts are dye-specific differences in electrophoretic mobility that can be obtained by calibration or estimated as part of base-calling, unless the electrophoretic data supplied to the basecaller has been preprocessed to correct for these shifts. Several algorithms for determining mobility shifts have been described, which typically conduct local searches in windowed time regions for the set of shifts that result in minimizing some measure of peak overlap between dye channels.

After the trace data has been obtained at 103, the sequence data processing tool manipulates the trace data to narrow the original peaks and reduce any overlap between peaks and thus, accomplish better peak segregation. Preferably, a sharp peak of zero width - a delta function in mathematical terms—would identify all, and now well-separated, peaks. In a preferred embodiment, this is accomplished by applying a cross-correlation computation of the current trace segment with an ideal, Gaussian—shaped peak.

Segments with peak characteristic, i.e. center of segment has maximal trace value will have high cross-correlation with the model peak (correlation coefficient r near +1), concave regions will have negative correlation (r˜−1), monotone regions will result in no correlation (r˜0). Multiplying the original trace with the corresponding value of r, that has been re-scaled to lie between 0 and 1, will in effect narrow peaks, and repeated application would arrive at delta functions. The cross-correlation transformation is accomplished in a single pass as follows:

f(base,loc)=R[base,loc]*T[base,loc] with R[base,loc]=(r(T[base,loc],MP)+1)/2 (1)

where T(base, loc) is the fluorescence intensity (trace value) detected for the color of the dye associated with base (A, C, G or T) at location loc; i.e., r( ) denotes the cross-correlation coefficient as explained below, and MP denotes the ideal Gaussian model peak.

Values R(base,loc) essentially provide a peak-shape indicator at all trace locations that is used later during base calling. The cross-correlation coefficient r is computed as:

$$r = ( 1 / ( N + 1 ) ) \\ {\sum }_{i} \{ \frac{( T [ base , loc - i ] - MP ( i ) ) * ( T [ base , loc - i ] - MP ( i ) )}{{σ}_{T} {σ}_{MP}} \} ; \\ with - 1 \leq r \leq + 1 ; and - N / 2 \leq i \leq + N / 2 \qquad ( 2 )$$

where σ_{T} and σ_{MP} are standard deviations of T and MP, respectively. N is the number of trace locations in the considered segment, preferably, N=6, i.e. a window of 7 trace points. If the number of trace points per initially assigned base call before quality filtering drops below 7, N is adjusted to N=4 to account for somewhat undersampled chromatograms. r is set to zero for both of the terminal 3 trace points.

The model peak is taken as an ideal Gaussian with:

$$MP ( i ) = \frac{1}{\sqrt{2 πσ}} exp ( - \frac{1}{2} {( \frac{i}{σ} )}^{2} ) \qquad ( 3 )$$

The standard deviation σ is set to 3.5 (2.5 for undersampled chromatograms according to the condition stated above).

At 105, the sequence data processing tool has generated four new traces that resemble the original traces, but have narrower peaks, i.e., the refined trace. At 107, these four traces are combined to produce one trace by essentially taking the maximum f-value at each trace location. In a closed form, and with some simultaneous smoothing, this new trace (termed “LT” or “Lifetrace” herein) is obtained by:

$$LT ( loc ) = \sqrt[k]{{\sum }_{bases} {f}^{k} ( base , loc )} with k = 4. \qquad ( 4 )$$

With larger values of k the value of LT(loc) converges to the maximum value of the four values off, while smaller values of k simultaneously smooth the function LT(loc). After testing a range of k-values, best results are obtained for k=4.

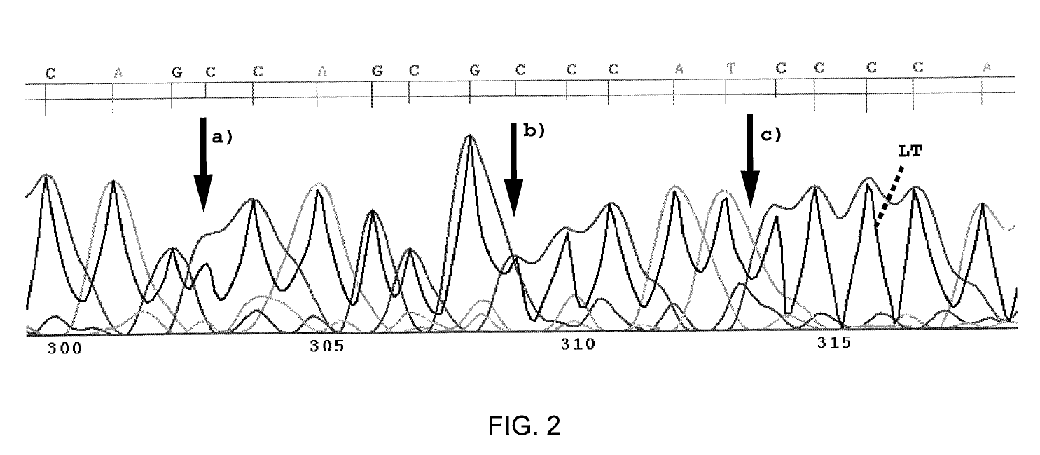

The described transformation process is illustrated in FIG. 2. Shown are the four original traces and the composite trace LT that provides the basis for peak detection. Basecalls are given in the top row with the length of the tick lines that indicate the peak location corresponding to the quality score with longer ticks indicating higher quality. The two horizontal lines mark quality score zero and 15. Locations a), b) and c) illustrate the facilitated peak detection provided by the trace transformations described herein (transformed trace LT) making it possible to reliably detect peaks that are peak shoulders and not local maxima, yet are real; to separate overlapping peaks; and to reduce noise from residual traces as they are not reflected in local maxima in the trace LT. It is evident that an improved peak separation is accomplished as is a reduction of noise. Instead of analyzing four traces to detect peaks, one trace (LT) is now sufficient. All local maxima and minima of LT are then detected by scanning through LT.

Peaks are identified as the middle data point of three consecutive data points wherein the inside data point is higher than the two outside data points (i.e., a local maxima method). Local minima (wherein the middle data point of three consecutive data points is lower than the two outside data points) are also identified. Alternatively, trace feature can be assigned as an actual peak whenever the difference between the maximum and an adjacent minimum exceeds a threshold value, e.g., 5%. A minimum peak height from the baseline may also be used to eliminate spurious peaks. Other peak detection methods are also possible and are well known in the art.

At 209, the actual base calling is conducted, i.e., the determined peaks are assigned a base. Basecalls are assigned to all detected local maxima of LT according to:

$$Base = {max}_{base = A , C , G , T} ( {S}_{base} ) \\ with {S}_{base} = R [ base , loc ] * A [ base , loc ] / {\sum }_{j = 1}^{4} A [ j , loc ] ) \qquad ( 5 )$$

where R(base,loc) are the peak shape factors obtained from Eq. 1, A is the area underneath a trace in a window of 7 trace pixels centered at loc. Effectively, the base with the maximal fractional area at a given peak location is chosen weighted by how peak-like the trace of a given base is (factor R). If the assigned base is the third or fourth base when traces are sorted according to decreasing fractional area at the current location alone (without factor R), an “N” (for not determined) is assigned to the current peak.

B. Calculation of Quality Scores

Equally important as the actual basecalls are associated quality scores that allow an assessment of the reliability of the call and to discriminate high-quality from low-quality calls. See, Lawrence et al. (1994) Nucl. Acid Res. 22: 1272-1280 and Ewing (1998) supra. The present invention distinguishes between two different quality scores: the quality of the call, and the quality of the space between calls (gap-quality) as an indication that a true base may not have been called.

The gap-quality score provides an estimate of the probability that a basecall has been missed, i.e., a probability that a deletion error has occurred during base calling. Use of the gap-quality score in the alignment process provides improved results by allowing accurate assignment of deletion errors during alignment. As such, the gap-quality may be used to identify deletion SNPs (Single Nucleotide Polymorphisms) where a potential base deletion needs to be distinguished reliably from a basecall error. Improved results can be achieved for virtually any method (e.g., assembling sequences into a consensus sequence, performing multiple sequence alignments to identify a motif, etc.) that utilizes sequence alignments through the use of the methods disclosed herein.

Moreover, deriving error statistics in conjunction with quality scores requires that basecall errors are located correctly during alignment. For example, prior standard dynamic programming often incorrectly assigned a deletion error to a high-quality basecall and not to an ambiguous trace location. Similarly, an insertion followed by a deletion a few bases later based on trace data could be misinterpreted as a single substitution error. The present methods provide for improved calibration of quality scores through the accurate determination of deletion errors.

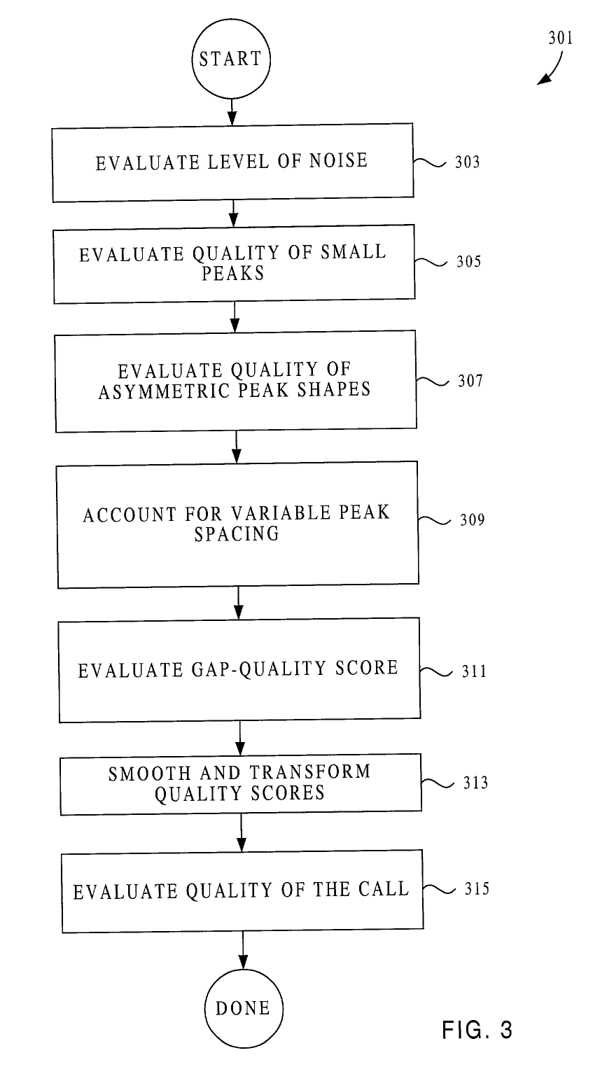

A high level process flow 301 for the computation of quality scores for the called bases in accordance with one embodiment of this invention is depicted in FIG. 3. The quality score of a base is calculated from the trace properties at and near its peak position. First, at 303, the level of noise, i.e. secondary peaks underneath the called base, is evaluated:

$$Q = \frac{{S}_{largest} - {S}_{secondlargest}}{{\sum }_{i = A , C , G , T} {S}_{i}} \qquad ( 6 )$$

where S is obtained from Eq. 5, and S_{largest} and S_{second largest} refer to the respective largest and second largest values of S.

At 305, quality scores associated with peaks smaller than one third of the mean peak height Pₘ of 20 base calls centered at the base are multiplied by sqrt(LT/loc)/(Pₘ/3)). For peaks with non-ideal peak shape, LT(loc) will be smaller than the maximal trace value at this position and, correspondingly:

$${Q}^{′} = Q * {( \frac{LT ( loc )}{{T}_{max}} )}^{2} \qquad ( 7 )$$

where Tₘₐₓ is the maximal trace value found at location loc.

At 307, asymmetric trace shapes of LT around basecalls where factored into Q by:

$${Q}^{″} = {Q}^{′} * \frac{r + 1}{2} \qquad ( 8 )$$

where r is the linear correlation coefficient between values of LT_{loc+i} and LT_{loc-i} with i running from 1 to integral value of half the mean peak separation; i.e. before and after the peak.

Variable peak spacing as an indicator of low quality is accounted for at 309 by:

$${Q}^{′′′} = \frac{{Q}^{″}}{exp ( 2 {σ}_{d} / ⟨ d ⟩ )} \qquad ( 9 )$$

where <d> denotes the mean peak spacing calculated for the first 20 peak-peak distances in the left and right neighborhood of a given call where both the call position and the following call positions have values of LT greater than one third of the LT associated with the current position, and σ_{d} is the associated standard deviation.

At 311, the gap-quality score is evaluated. The gap-quality score is composed of two components: the degree of noise between two consecutive calls, and overly wide peak spacing between bases i and i+1 indicative of another base that might be there but was not called:

Q_{gap}=(1−Rₙₒᵢₛₑ) (10)

if(d_{i,i+1}><d>)Q_{gap}=Q_{gap}*(<d>/d_{i,i+1})^{1/max(0.1,R}^{ₙₒᵢₛₑ}) (11)

where Rₙₒᵢₛₑ is the fractional area of alternate base traces under the called peaks i and i+1 If a base is removed during quality filtering, the gap quality score of the base preceding this call is lowered. The last base call is assigned an arbitrary gap-quality score of 0.5 (note that scores are re-scaled later).

As a last processing step, at 313, the quality scores are smoothed across all basecalls, and transformed in scale to adhere to the convention that q=−10×log₁₀(p) (Ewing (1998) supra) where q is the quality score, and p is the true observed error rate. Since the quality scores yielded a monotonic q to p relationship resembling a quadratic function in the semi-logarithmic plot, scale calibration was accomplished by a simple transformation. If a q-score of a given base is greater than the q-score of the preceding and following basecall, it is re-calculated as the arithmetic mean of the three. This was implemented to avoid high q-scores in otherwise low-quality regions.

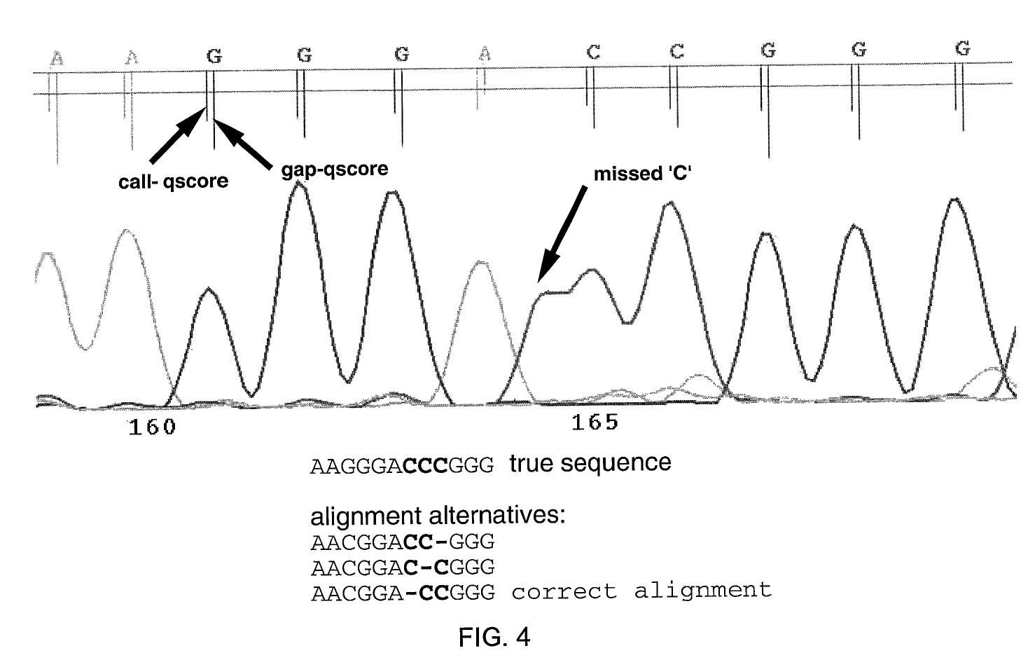

FIG. 4 exemplifies the concept of a gap-quality score. In this example, a basecall error has occurred: a true ‘C’ basecall is missed. This single C-deletion can generate three different alignments of equal alignment score shown below. However, the chromatogram suggests that the error has occurred in the first position of the three ‘C’ run. This is reflected in the low gap-quality score of the preceding ‘A’ as compared to the high quality scores of the neighboring basecalls. By taking into account gap-quality scores during alignments, the gap is correctly positioned at the first position. FIG. 4 also illustrates how a deletion error in a run of the same base can be aligned differently. The gap-quality scores help locate the deletion error and the link between gap-quality score and deletion error can be established correctly.

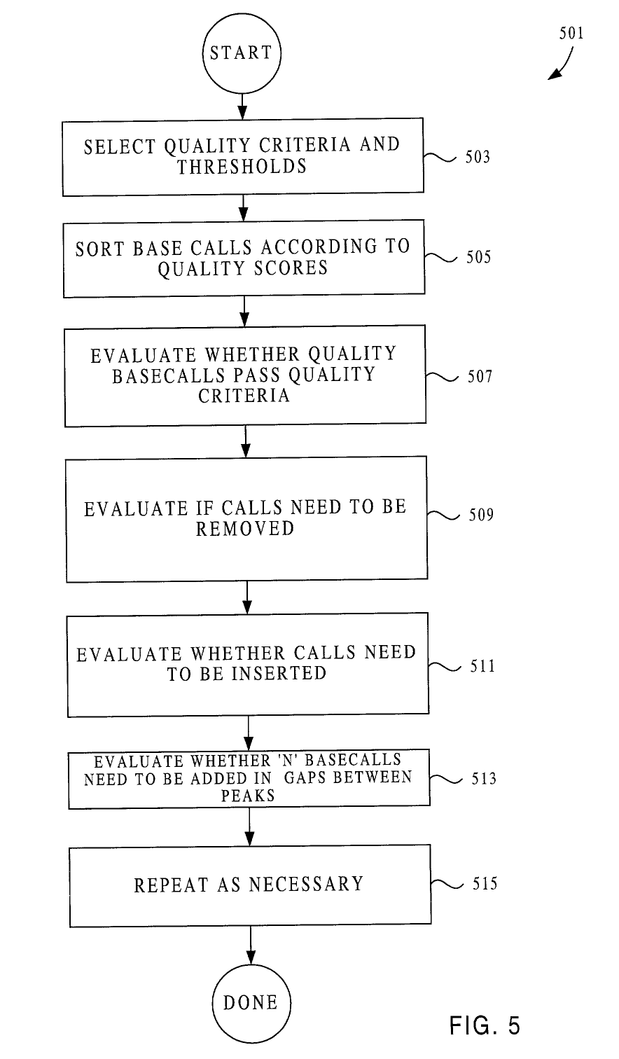

FIG. 5 illustrates a high level process 501 for the performance of quality filtering on the called bases. Preferably, two iterations of quality filtering are performed in which, according to several quality criteria, peaks can be removed or merged in cases of runs of the same base. Finally, traces are checked for possible basecall additions in cases of broad peaks where the peak detection algorithm may have assigned too few bases.

The selection of quality criteria and associated quality thresholds used during quality filtering can be derived heuristically. See 503. One such parameter for quality filtering is the proper estimation of the correct peak spacing. The present invention attempts to infer the correct peak to peak distances in regions of low trace data quality from the closest—in terms of location—available regions of higher quality as determined by the internally assigned quality scores and uniformity of peak to peak distance in this region.

At 505, basecalls are sorted according to ascending order of quality score. At 507, starting with lowest quality, basecalls are checked whether they pass the imposed quality criteria and removed otherwise. These quality thresholds (generally nine or so thresholds are used) impose restrictions on the minimally acceptable peak height and peak to peak spacing before and after a potential basecall removal, and combinations thereof.

If a merger of two consecutive bases of the same type results in a new peak spacing that is more in line with higher quality regions and the corresponding trace between the two calls does not show a clear separation, the call with lower quality is removed. See 509.

Broad, but Gaussian-like peaks will initially get assigned a single basecall. However, it is possible that several bases of the same type are merged into one peak. To detect such peaks, at 511, the widths of all peaks are determined and then compared to the mean observed peak separation for high quality regions proximal to the current peak. If the integral value of the expression 0.45+peak₁₃width/peak₁₃spacing is greater than 1, a corresponding number of bases are added to the current peak. The width is determined by requiring that peaks of different bases do not overlap. Where the maximal trace value changes from one base to another, the value of LT drops below max(LTₛ)/10, or the maximal trace value at the current position drops below max(LTₛ)/6, the previous peak ends. The next peak starts where all the previously described thresholds are exceeded again. The index s denotes which of three equally sized segments of the chromatogram is currently being processed. This is done to account for changing maximal trace values across the chromatogram length. Inserted peaks are assigned an arbitrary quality score of max(Qscores)/10.

The peak width determination procedure also identifies gaps as the space in between peaks. For a variety of reasons, these gaps can represent real base drop-outs and a corresponding number of ‘N’-basecalls can be added.

C. Benchmarking Protocol

The present invention also provides a method for benchmarking the performance of base calling algorithms. More specifically, for testing the performance of the present invention and comparing it to phred, two different strategies were applied. In the first, referred to as Method 1, the benchmarking algorithm detailed in the original phred publication (Ewing et al. supra) was adopted. Here, the basecalls are aligned to the known true consensus sequence using cross₁₃match with alignment parameters as given in Ewing et al. supra. The alignment region where both called sequences can be aligned (i.e., the jointly alignable region) is analyzed for basecall errors; i.e. substitution errors, deletion errors, or insertion errors. Basecalls that go beyond the jointly alignable region and align to the true sequence are captured in the number of additionally aligned bases for the basecaller that generated these calls. In effect, this method confines the analysis to higher quality regions as both basecallers agree to large extent and, consequently, the error statistics have to be rather similar. It is possible, however, that one basecaller consistently generates more alignable bases with few basecall errors. In Method 1, this would be reflected by the number of additionally aligned bases, but would not allow a comparison of actual error rates in those regions.

In contrast to Method 1, where a consensus alignment is analyzed, error statistics are collected over the consensus sequence stretch whose boundaries are determined by the left-most (with regard to the consensus sequence) and right-most Blast High Scoring Pair (HSP) bounds (aligned segment between query (LifeTrace or phred) and consensus sequence) contributed by either basecaller in the methods described herein (i.e., Method 2). The rationale is that a high scoring Blast hit by either one of the two basecallers proves that the trace data permitted such accurate base calling, and therefore, the other basecaller underperformed.

For every chromatogram, the phred- and LifeTrace-generated nucleotide sequences were aligned to the consensus (true) sequence using the program blastn with default parameters (Altschul et al. (1990) J. Mol. Biol. 215: 403-410, version 2.0a19-WashU). The smallest and largest trace location associated with the first and the last base belonging to the top high scoring pairs (HSP) with ap-value smaller than 10⁻²⁰ from either the phred sequence, or LifeTrace sequence was used to determine the start and end location of “alignable” trace data. All bases falling in between the start and end trace location are excised out of both phred and LifeTrace sequences and then re-aligned using full dynamic programming to the determined hit region in the consensus sequence (sequence between the first and last consensus base found by either phred or LifeTrace). See, Needleman and Wunsch, (1970) J. Mol. Biol. 48: 443-453. To avoid attributing basecall errors to vector sequence, it was required that either basecaller had an exact match over at least 10 consecutive bases at both ends, and error statistics were collected only for the remaining middle section of the alignment.

Deriving error statistics in conjunction with quality scores requires that basecall errors are located correctly during alignment. For example, if a deletion error occurred in a run of 4 ‘C’s, where only 3 ‘C’s were called, the error could be attributed to any of the four bases not changing the global alignment score. It is therefore possible that such a deletion error is assigned incorrectly to a high-quality basecall during standard dynamic programming and not to an ambiguous trace location. Similarly, what in reality is an insertion followed by a deletion a few bases later based on trace data could be misinterpreted as a single substitution error. See, Bemo (1996) Genome Res. 6: 90-91. To diminish the impact of such problems, the actual quality scores as match scores and gap penalty during alignment were used. As a result, deletions in runs are placed at positions of lowest quality, i.e. the most likely place where the error has occurred, and matches are assigned with preference given to high quality base calls. In detail, a score of +1+LifeTraceQscore(baseCall)/5 for position specific matches, −2 for mismatch, −(3+LifeTraceGapQscore(baseCall)/10) as the position dependent gap penalty was used. Substitution and insertion errors are linked to the regular quality score of the corresponding basecall, and deletion errors are associated with the gap quality score of the base preceding the gap as it measures the quality of the gap to the next called base.

D. Software/Hardware

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read- only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

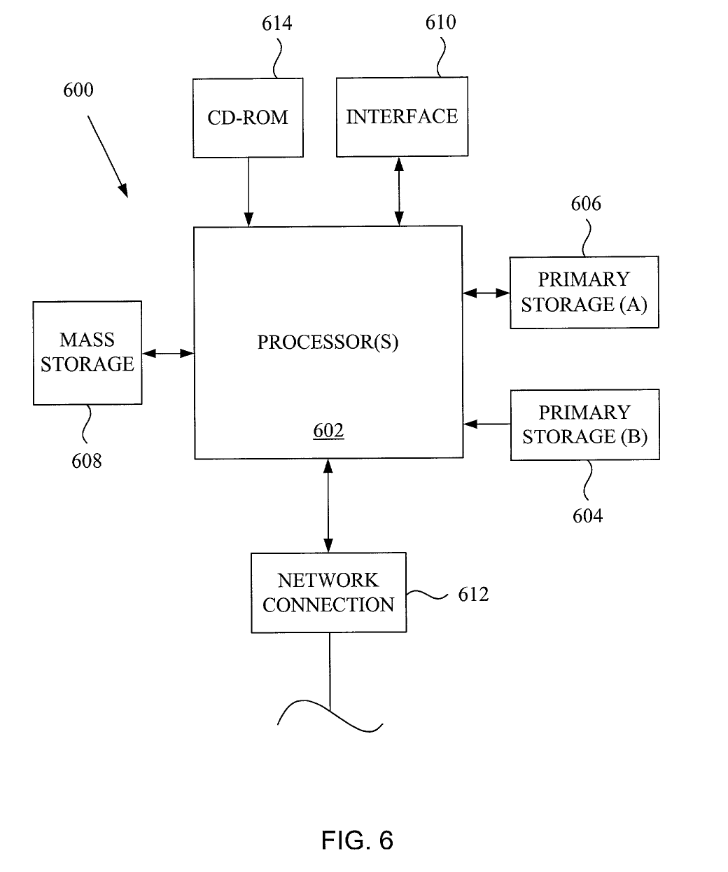

FIG. 6 illustrates a typical computer system that, when appropriately configured or designed, can serve as an image analysis apparatus of this invention. The computer system 600 includes any number of processors 602 (also referred to as central processing units, or CPUs) that are coupled to storage devices including primary storage 606 (typically a random access memory, or RAM), primary storage 604 (typically a read only memory, or ROM). CPU 602 may be of various types including microcontrollers and microprocessors such as programmable devices (e.g., CPLDs and FPGAs) and unprogrammable devices such as gate array ASICs or general purpose microprocessors. As is well known in the art, primary storage 604 acts to transfer data and instructions uni-directionally to the CPU and primary storage 606 is used typically to transfer data and instructions in a bi-directional manner. Both of these primary storage devices may include any suitable computer-readable media such as those described above. A mass storage device 608 is also coupled bi-directionally to CPU 602 and provides additional data storage capacity and may include any of the computer-readable media described above. Mass storage device 608 may be used to store programs, data and the like and is typically a secondary storage medium such as a hard disk. It will be appreciated that the information retained within the mass storage device 608, may, in appropriate cases, be incorporated in standard fashion as part of primary storage 606 as virtual memory. A specific mass storage device such as a CD-ROM 614 may also pass data uni-directionally to the CPU.

CPU 602 is also coupled to an interface 610 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to an external device such as a database or a computer or telecommunications network using an external connection as shown generally at 612. With such a connection, it is contemplated that the CPU might receive information from the network, or might output information to the network in the course of performing the method steps described herein.

In one embodiment, the computer system 600 is directly coupled to an electrophoresis detection instrument. Data from the electrophoresis detection instrument are provided via interface 612 for analysis by system 600. Alternatively, the data or traces processed by system 600 are provided from a data storage source such as a database or other repository. Again, the images are provided via interface 612. Once in the computer system 600, a memory device such as primary storage 606 or mass storage 608 buffers or stores, at least temporarily, the data or trace images. With this data, the image analysis apparatus 600 can perform various analysis operations such as base calling, benchmarking and the like. To this end, the processor may perform various operations on the stored images or data.

### EXAMPLES

The following examples provide the experimental results illustrating the effectiveness of methods and systems in accordance with the present invention for base calling and benchmarking. It should be understood the following is representative only, and that the invention is not limited by the detail set forth in these examples.

General

The phred version 0.99077.f was used in this study. This version of phred utilizes instrument-specific quality score calibrations for ABI-377, MegaBACE 1000, ABI-3700.

LifeTrace was written in C. It provides a graphical interface to display chromatogram trace data based on the standard X11 library and should run on any UNIX Xwindow system.

A. Performance Testing

Performance of the presents methods was evaluated for three commonly used sequencing machines: MegaBACE 1000 and ABI-3700 capillary sequencers, and the ABI-377 slab gel sequencing machine. Large sets of MegaBACE reads from three human BAC clones (chromosome 7) for accuracy assessment of the present invention (“LifeTrace”) and phred base-callers shown below in Table 1 were used.

TABLE 1
BAC Clone Description

| | | | | | | ID in |
|---|---|---|---|---|---|---|
| | Accession | | | | | this |
| BAC clone | # | GI # | Size | Reads | Chemistry | paper |
| RP11- | AC00731 | 4586080 | 185652 | 8273 | Dye Primer | MB_pri |
| 349E11 | 2 | | bp | | | m |
| RP11- | AC00954 | 6554502 | 160367 | 3264 | ET | |
| 260N14 | 2 | | bp | | Terminator | MB_ter |
| RP11- | AC00917 | 6642684 | 178097 | 3360 | ET | m |
| 169C22 | 8 | | bp | | Terminator | |

Each of these clones was shotgun sequenced to high depth (10x-20x). The sequences were then assembled and finished. The accuracy of the finished sequences is very high—probably less than 1 error in 50,000 bases. Thus these sequences are suitable for evaluating base-caller accuracy.

Table 2 below shows the number of reads used in the analysis. Sequences were read using Amersham's MegaBACE 1000 capillary sequencer. Trace processing was done using the Cimarron v1.61 analysis software (Cimarron Software Inc., Salt Lake City, Utah). The data sets are naturally grouped by chemistry so dye primer reads were analyzed separately from dye terminator reads. Additional testing was performed for a total of 4,714 ABI-3700 sequencer chromatograms of mixed chemistry (primer, terminator). A small set of 1,184 ABI-377 chromatograms that assemble into Human Collagenase (GenBank Accession number: U78045) was used for benchmarking the slab gel sequencer.

B. Benchmarking

The benchmark statistics for the two basecallers phred and LifeTrace obtained from performance testing according to Method 1 (see section Performance Testing) for the MegaBACE chromatogram sets are presented in Tables 2 and 3 below. The present invention provides for 2.4% more aligned bases than phred for dye primer and 2.1% more for dye terminator. The bulk of this difference comes from longer reads but a significant fraction also comes from additional aligned reads.

TABLE 2
Alignment results.

| | |
|---|---|
| MB_Prim | MB_Term |

| | Aligned | Aligned | Aligned | Aligned |
|---|---|---|---|---|
| Basecaller | reads | bases | reads | bases |
| Phred | 5299 | 2425026 | 5231 | 2639830 |
| LifeTrace | 5352 | 2483208 | 5292 | 2696119 |

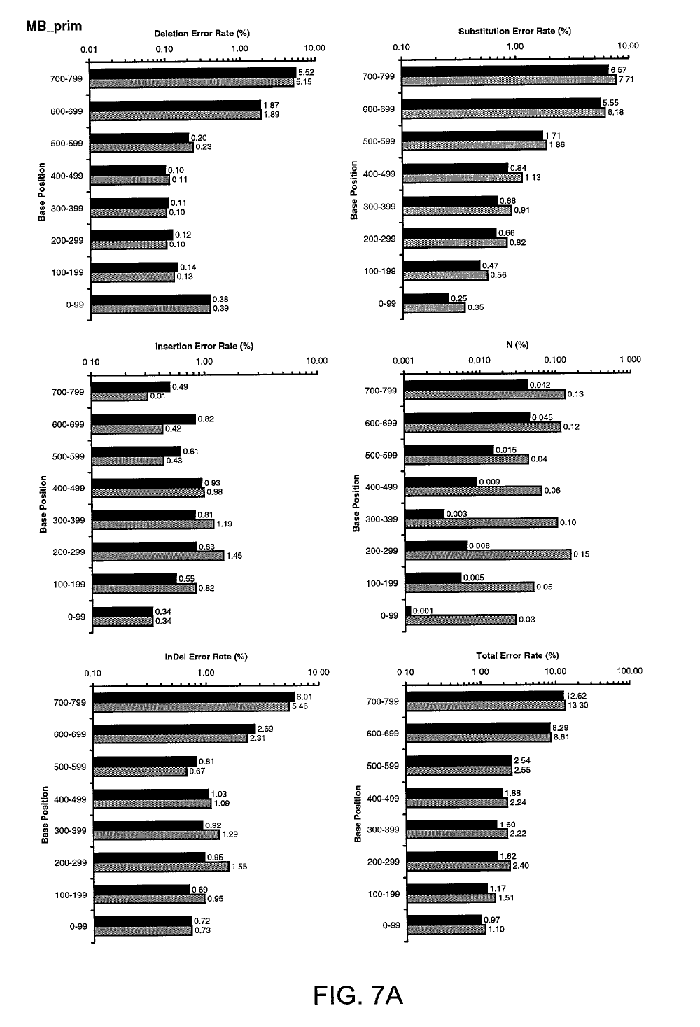

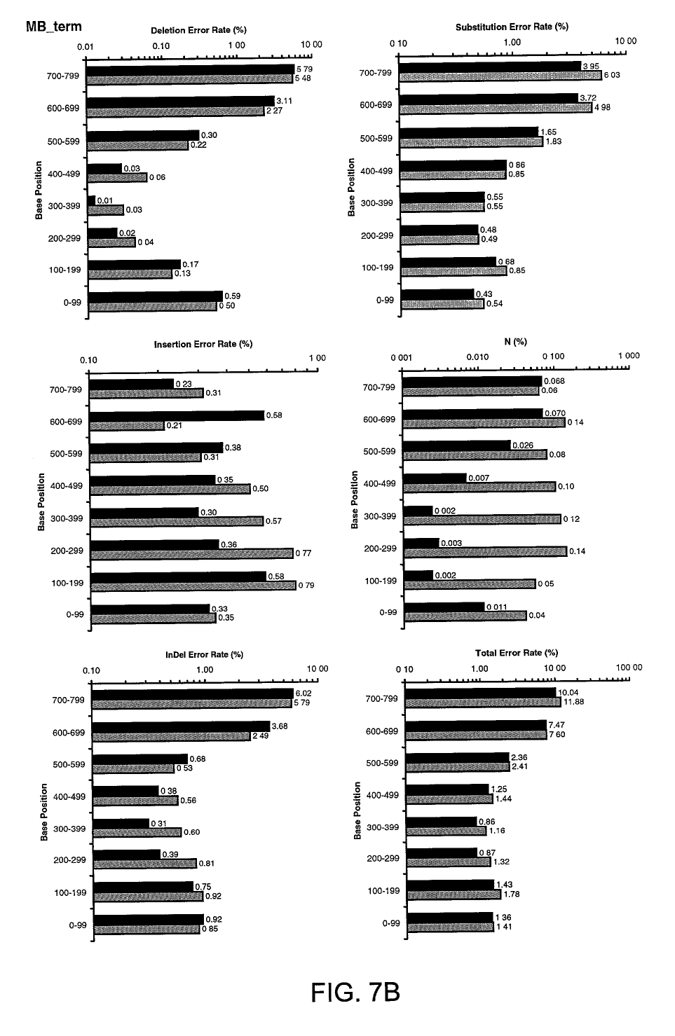

Overall the present methods made 17% fewer errors for dye primer data with 17% fewer substitution errors and 16% fewer indels. For dye terminator data, the present methods made 13% fewer errors overall with 15% fewer substitution errors and 10% fewer indels. The break-down per error type and base position is given in FIG. 7. For both sets, dye-primer and dye-terminator, and for all position ranges the methods described herein generate consistently fewer total errors, calls fewer ‘N’s, and makes fewer substitution errors. The number of indels generated by the methods described herein (insertions and deletions combined) is lowered significantly in the range of base position 100-500, the range that usually contributes the most high-quality trace information and the most base calls in the error statistics (see Table 3).

TABLE 3
The total number of jointly aligned bases by read position and
chemistry

| Base Position | MB_Prim | MB_Term |
|---|---|---|
| 0-99 | 168823 | 175661 |
| 100-199 | 498926 | 501383 |
| 200-299 | 449075 | 484530 |
| 300-399 | 397832 | 458358 |
| 400-499 | 359640 | 428983 |
| 500-599 | 298010 | 367775 |
| 600-699 | 159247 | 177021 |
| 700-799 | 14987 | 7941 |
| 800-899 | 169 | 27 |
| 900-999 | 6 | |

By restricting the error analysis to regions where both basecallers align to the true sequence, Method 1 will tend to gather error statistics for regions where both basecallers generate few errors. It is possible, however, that what is given as additionally aligned bases in Method 1 for the present methods are in fact high-confidence base calls with few errors for a region where phred introduces exceptionally many errors. For example, for a particular chromatogram, Method 1 generated a jointly alignable sequence region of 202 bases with 7 errors for phred and zero errors for the present methods with 264 extra aligned bases. By contrast, Method 2 generates an initial blast alignment of 465 bases based on LifeTrace-called sequence with 67 base call errors in the equivalent chromatogram region by phred and zero by the methods described herein. Evidently, Method 2 widens the performance difference by further analyzing the extra aligned bases.

The performance comparison between the basecallers phred and the methods described herein using Method 2 is summarized in Table 4.

MB_prim
Total base calls aligned: 2,404,898

| Phred | | | | | Total |
|---|---|---|---|---|---|
| LifeTrace | Correct | Subst | Insert | Del | LifeTrace |
| Correct | 2,346,881 | 12,192 | 43,884 | 8,508 | |
| Subst | 10,7271 | 14,069 | 0 | 2,232 | 27,028 |
| Insert | 21,300 | 0 | 6,072 | 0 | 27,372 |
| Del | 4,836 | 1,179 | 0 | 6,072 | 12,087 |
| Total Phred | | 27,440 | 49,956 | 16,812 | |

Summary:
Both correct: 97.6% of all aligned true-sequence bases
Total LifeTrace errors: 64,689 = 70% of
Phred errors, Total Phred errors: 92,410
Total InDels LifeTrace: 37,661 = 57.9%
of Phred InDels, Total Phred: 64,970
Mean Blast hit length to true consensus
sequence, LifeTrace: 517.5, Phred: 493.9
MB_term
Total base calls aligned: 2,748,823

| Phred | | | | | Total |
|---|---|---|---|---|---|
| LifeTrace | Correct | Subst | Insert | Del | LifeTrace |
| Correct | 2,691,854 | 11,020 | 33,532 | 8,049 | |
| Subst | 10,770 | 15,215 | 0 | 1,434 | 27,419 |
| Insert | 11,573 | 0 | 3,609 | 0 | 15,182 |
| Del | 6,714 | 1,477 | 0 | 2,290 | 10,481 |
| Total Phred | | 27,712 | 37,141 | 11,773 | |

Summary:
Both correct: 97.9% of all aligned true-sequence bases
Total LifeTrace errors: 53,082 = 69.2%
of Phred errors, Total Phred errors: 76,626
Total InDels LifeTrace: 25,663 = 52.3%
of Phred InDels, Total Phred: 48,914
Mean Blast hit length to true consensus
sequence, LifeTrace: 532.3, Phred: 517.5
377
Total base calls aligned: 666,489

| Phred | | | | | Total |
|---|---|---|---|---|---|
| LifeTrace | Correct | Subst | Insert | Del | LifeTrace |
| Correct | 644,389 | 5,612 | 2,974 | 1,843 | |
| Subst | 4,414 | 6,865 | 0 | 721 | 12,000 |
| Insert | 4,424 | 0 | 651 | 0 | 5,075 |
| Del | 1,671 | 317 | 0 | 657 | 2,645 |
| Total Phred | | 12,794 | 3,625 | 3,221 | |

Summary:
Both correct: 96.7% of all aligned true-sequence bases
Total LifeTrace errors: 19,720 = 100.4%
of Phred errors, Total Phred errors: 19,640
Total InDels LifeTrace: 7,720 = 113.2%
of Phred InDels, Total Phred: 6,846
Mean Blast hit length to true consensus
sequence, LifeTrace: 582.6, Phred: 594.2
3700
Total base calls aligned: 2,659,195

| Phred | | | | | Total |
|---|---|---|---|---|---|
| LifeTrace | Correct | Subst | Insert | Del | LifeTrace |
| Correct | 2,519,021 | 31,671 | 23,497 | 17,676 | |
| Subst | 17,493 | 20,863 | 0 | 2,698 | 41,054 |
| Insert | 11,930 | 0 | 1,482 | 0 | 13,412 |
| Del | 34,113 | 5,257 | 0 | 10,403 | 49,773 |
| Total Phred | | 73,397 | 24,979 | 30,777 | |

Summary:
Both correct: 94.7% of all aligned true-sequence bases
Total LifeTrace errors: 104,239 = 91.8%
of Phred errors, Total Phred errors: 113,547
Total InDels LifeTrace: 63,185 = 113.5%
of Phred InDels, Total Phred: 55,756
Mean Blast hit length to true consensus
sequence, LifeTrace: 662.5, Phred: 705.8

More specifically, Table 4 shows a break-down of error statistics derived from testing the performance using Method 2 (see methods) applied to both the MegaBACE dye-primer and dye-terminator set. Table lists all possible error combinations. For example, for the set MB₁₃prim there were 12,192 correct calls made by LifeTrace where phred had a substitution error at the same position compared to 10,727 where phred was correct and LifeTrace had a substitution error and 14,069 cases where both basecallers had a substitution error. ‘Mean Blast hit length’ refers to the length of the high scoring sequence alignment between the called sequence and the finished, true consensus sequence. Called ‘N’s are counted as bases and contributed to substitution and insertion errors.

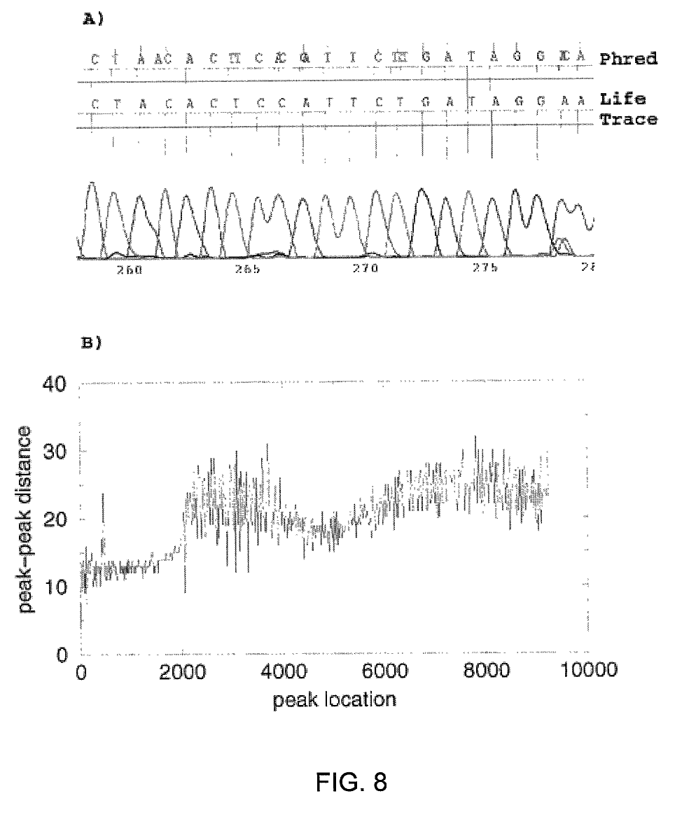

For the two MegaBACE sets (dye-primer and dye-terminator) LifeTrace overall generates about 30% fewer basecall errors than phred. As explained above, this sharper decrease of errors generated by LifeTrace compared to phred in Method 2 compared to Method 1 originates from extended error analysis into the extra aligned bases by LifeTrace. Insertion errors in particular are reduced significantly. This can be attributed to the frequent failure of phred to adjust to variable peak-spacing as illustrated in FIG. 8. The number of substitution errors by LifeTrace is also reduced compared to phred. For the primer set, there are 12,192 basecalls where phred has a substitution error and LifeTrace is correct, contrasted by only 10,727 (12% fewer) cases for which phred is correct and LifeTrace miscalled a base. The decrease of substitution errors for the same comparison is 2.3% for dye-terminator data. The total number of indels produced by LifeTrace is significantly lower (42% less for the dye-primer, and 47% less for the dye-terminator set) largely because of a much reduced number of insertion errors. LifeTrace generated on average 3-5% longer initial Blast alignments of the called sequence to the true sequence than phred indicative of the increased number of correct calls.

For the ABI-377-sequencer chromatogram set the overall performance is comparable with almost exactly the same overall error rates for phred and LifeTrace. The break down into error types reveals that LifeTrace generates more insertion and deletion errors for this set, offset by a reduced number of substitution errors. The higher number of indels (insertions and deletions) is also reflected in 2% shorter initial Blast-alignments of the called sequence to the true consensus. It needs to be noted, however, that indels are more critical in the context of sequence assemblies where indels are more difficult to deal with than substitution errors and can cause severe frameshift errors.

Similar results were obtained for ABI-3700 chromatograms for which LifeTrace generated 29% fewer substitution errors, but 13% more indels with an overall decrease of errors of about 10%. The relative increase of basecall errors of LifeTrace compared to phred was largely confined to the end of the reads; i.e. in very low quality regions. When the reads were clipped off at pixel position 6000 corresponding to a read length of about 500 nucleotides or about two thirds of the original length, the error statistics are much more in favor of LifeTrace with 6% fewer substitution errors, 20% fewer indels, and 13% fewer errors overall. Thus, while LifeTrace generated more errors in the low quality terminal read segments, it produced significantly fewer errors in the higher quality parts. Many post-processing steps include some sort of quality clipping so the reduced number of errors in the higher quality parts is even more significant.

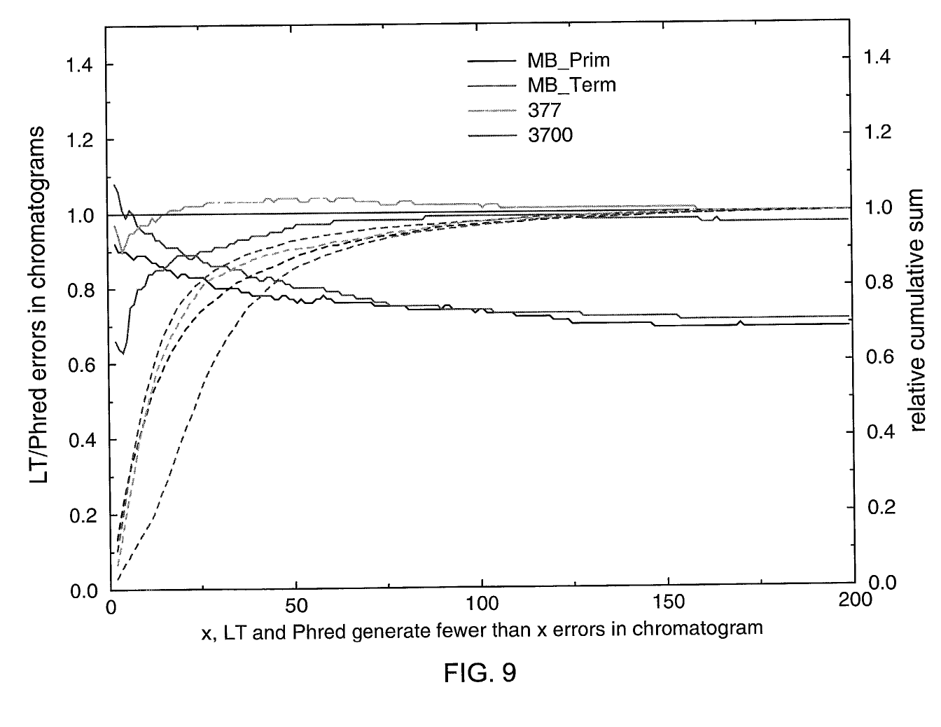

The substantial reduction of MegaBACE basecall errors achieved by LifeTrace is largely attributable to chromatograms for which phred introduces exceptionally many errors. FIG. 9 shows the LifeTrace error rate relative to phred as a function of errors detected in the chromatogram by the larger error number of either phred or LifeTrace. The improved performance of LifeTrace is more pronounced for chromatograms with many errors (>25). Again, this can be explained by the observed difficulties of phred to adjust to variable peak spacing. Many of these chromatograms appear to have high quality, yet phred inserts additional bases to maintain uniform peak spacing (FIG. 8). However, LifeTrace also outperforms phred in higher quality chromatograms where both basecallers generate few errors. Only for dye-terminator chromatograms with very few errors (<6 errors) does LifeTrace produce slightly more errors (about 5%). However, this subset of chromatograms includes only about 20% of all chromatograms analyzed as can be seen from the cumulative chromatogram counts in FIG. 9. The comparison of LifeTrace to phred is nearly flat for ABI-377 data suggesting that both basecallers perform uniformly over all chromatogram quality ranges. Contrary to MegaBACE data, there appears to be a performance gain from LifeTrace in higher quality chromatograms from the ABI-3700. LifeTrace is observed to cause fewer errors in chromatograms where both LifeTrace and phred make relatively few errors. This is in line with the reduced error rates for clipped ABI-3700 chromatograms described above.

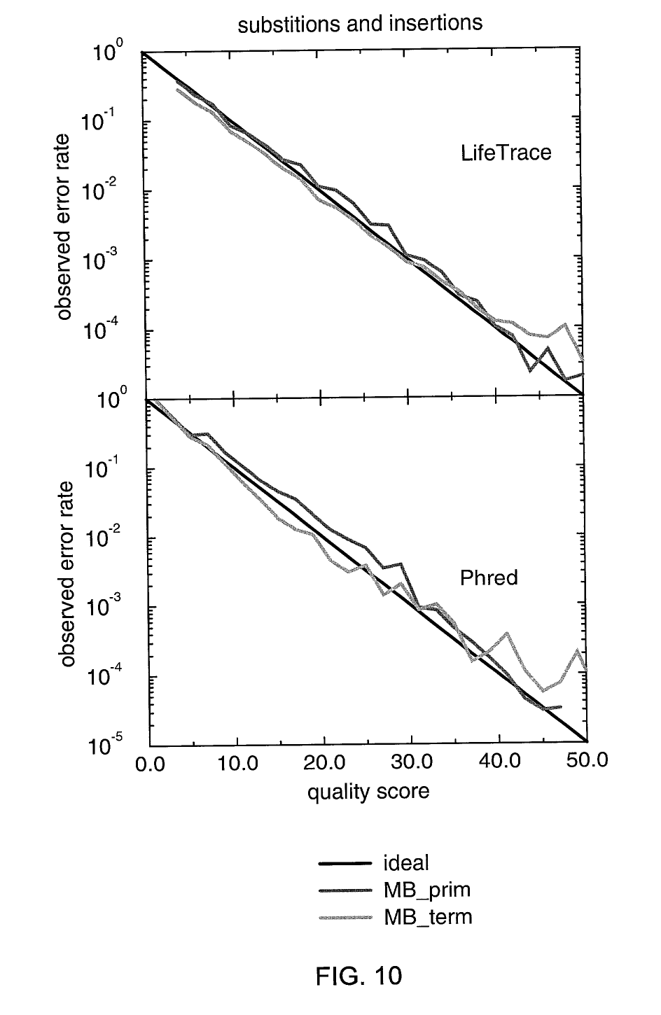

LifeTrace distinguishes between two quality scores: the quality of an actual basecall, and the quality of the gap between bases. As the trace-related parameters influencing the LifeTrace quality scores generated raw quality scores that showed a monotonic relationship with the true observed error rate, it was possible to calibrate both the basecall quality score and the gap quality score to the convention introduced by phred where q=−10×log₁₀(error rate). The calibrated quality scores assigned to the called bases are compared to the observed error rate in FIG. 10. For both sets, primer and terminator, the LifeTrace quality scores prove to be reliable predictors of the expected error rate and fall within a narrow range from the ideal line; similarly for phred, albeit the spread between the two sets is somewhat wider. It has to be noted, however, that phred quality scores estimate the probability of all three error types: substitutions, insertions, and deletions. Deletion errors were not considered in FIG. 6, neither for LifeTrace nor for phred. A deleted base cannot have an associated quality score. The present invention introduce the gap-quality score, whereas phred propagates low quality gaps (wide gaps, or gaps with potential peaks in between) to quality scores of the neighboring basecalls.

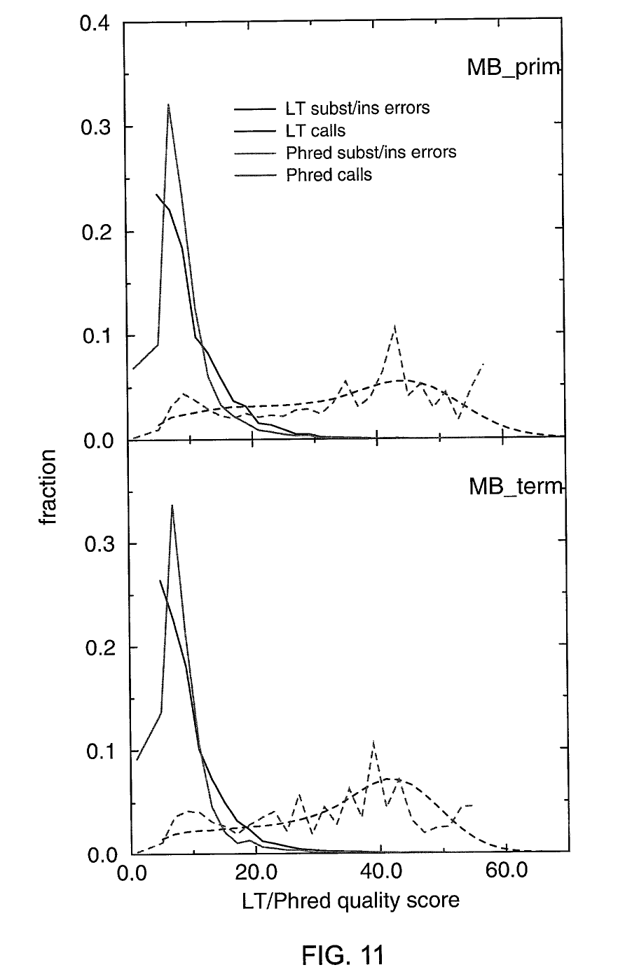

An objective of base calling by means of assigning quality scores is to reliably separate high-quality bases from potentially incorrect basecalls. FIG. 11 plots the frequency histogram for the quality scores associated with basecall errors compared to the distribution of quality scores for all calls for LifeTrace and phred. As desired, basecall errors accumulate in low-quality regions and are well separated from the majority of basecalls. While the overall distribution is similar for LifeTrace and phred, the histogram for phred is much more rugged. This is an effect introduced by the lookup-table approach taken by phred to match trace parameters with quality scores/observed error rates. Instead, LifeTrace uses continuous parameters to judge quality, and therefore the curves appear smoother.

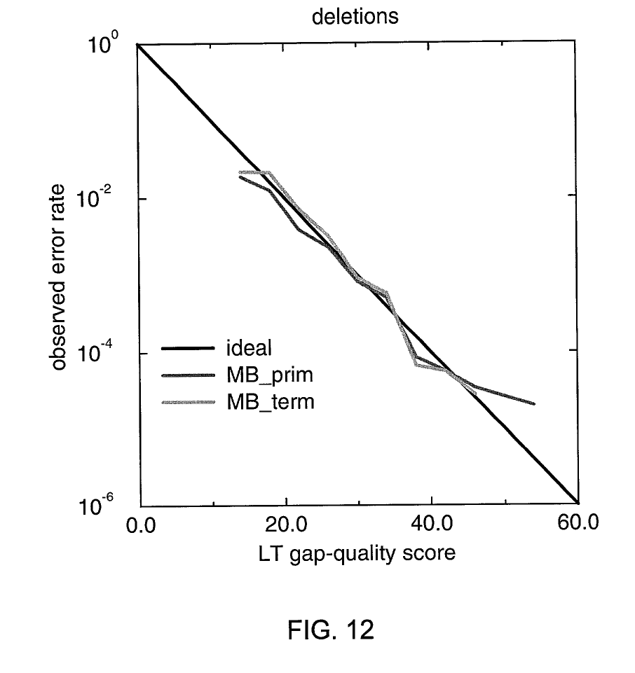

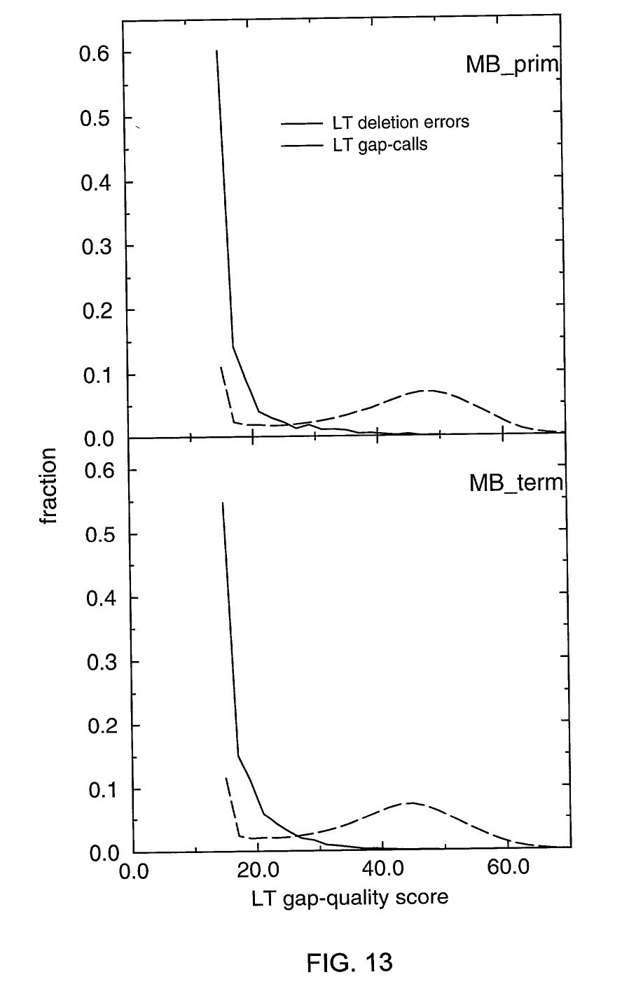

FIG. 12 shows that the assigned gap-quality scores have predictive value and correctly estimate the observed error rate. Deletion errors are confined to low gap-quality gap-calls, well separated from the bulk of higher quality data (FIG. 13). FIGS. 12 and 13, showing data for deletion errors, are the equivalent plots to FIG. 10 and 11 for the substitution/insertion error category. In the current implementation, the lowest possible gap-quality scores is 15 because of a single particular threshold in one of the components contributing to the gap-quality. As many gap-calls actually fall below that, counts at gap-quality=15 are elevated.

It remains to be noted that the accuracy of base calling is also influenced to large degree by the pre-processing applied to the chromatograms and changes in the pre-processing steps will result in different comparison results. Other technical parameters, as for example, the chosen read length or sampling rate per peak systematically influence the quality of the recorded chromatogram and renders chromatogram sets different even if produced on the same machine type. Preferably, such systematic differences between sets will be accounted for by a calibration of quality scores.

### Software Appendix

The Software appendix which is included as part of the specification (copyright, Incyte Genomics, Inc.) provides pseudocode code for implementation of an embodiment of the present invention. pseudocode for implementation of the invention. However, it should also be noted that there are alternative ways of implementing the invention.

The above description is illustrative and not restrictive. Many variations of the invention will become apparent to those of skill in the art upon review of this disclosure. Merely by way of example, while the invention is illustrated with particular reference to the evaluation of DNA (natural or unnatural), the methods can be used in the analysis of other materials, such as RNA. The scope of the invention should, therefore, be determined not with reference to the above description, but instead should be determined with reference to the appended claims along with their full scope of equivalents.

What is claimed is:

1. A method of determining a sequence of a nucleic acid polymer, comprising the steps of:
(a) obtaining data traces from a plurality of channels of an electrophoresis detection apparatus, each channel detecting the products of a DNA sequencing reactions;
(b) combining the data traces by a process comprising the steps of:
(i) applying a cross-correlation coefficient to each of the four data traces to yield four refined traces, wherein the cross-correlation coefficient compares each of the traces with an ideal, Gaussian-shaped peak and wherein the refined traces have narrower peaks than the corresponding data traces;
(ii) combining the four refined traces to yield a composite trace;
(c) detecting peaks in the composite trace by a process that is independent of peak spacing; and
(d) determining the sequence of the nucleic acid polymer by assigning basecalls to the peaks.

2. The method of claim 1, wherein the data traces have been preprocessed.

3. The method of claim 2, wherein preprocessing comprises the steps of:
(i) obtaining unprocessed data traces from a plurality of channels of an automated electrophoresis detection apparatus, each channel detecting the products of a DNA sequencing reactions;
(ii) identifying begin and end points in the unprocessed data;
(iii) establishing a baseline in the unprocessed data,
(iv) subtracting the baseline from the unprocessed data to generate the baseline-corrected data; and
(v) separating the baseline-corrected data to generate the data traces, the separating step comprising spectral or leakage separation.

4. The method of claim 1, wherein the electrophoresis detection apparatus uses slab gel electrophoresis; tube gel electrophoresis; or capillary gel electrophoresis.

5. The method of claim 4, wherein the electrophoresis detection apparatus is a MegaBACE capillary sequencing machine.

6. The method of claim 1, further comprising the step of generating at least one quality score for at least one basecall.

7. The method of claim 6, wherein the at least one quality score is a gap-quality score, wherein the gap-quality score estimates the probability of a deletion error between two adjacent assigned basecalls.

8. The method of claim 7, wherein the gap-quality score measures degree of noise between the two adjacent assigned basecalls and overly wide peak spacing between the two adjacent assigned basecalls.

9. The method of claim 6, further comprising the step of using the quality scores for quality filtering whereby basecalls can be removed or added from the sequence of the nucleic acid polymer during the quality filtering.

10. The method of claim 1, wherein the DNA sequencing reactions utilize dye-terminator or dye-primer chemistry.

11. A computer program product comprising a machine readable medium on which is provided program instructions for determining a sequence of a nucleic acid polymer, the instructions comprising:
code for obtaining data traces from a plurality of channels of an electrophoresis detection apparatus, each channel detecting the products of a DNA sequencing reactions;
code for combining the data traces by a process comprising the steps of:
(i) applying a cross-correlation coefficient to each of the four data traces to yield four refined traces, wherein the cross-correlation coefficient compares each of the traces with an ideal, Gaussian-shaped peak and wherein the refined traces have narrower peaks than the corresponding data traces;
(ii) combining the four refined traces to yield a composite trace;
code for detecting peaks in the composite trace by a process that is independent of peak spacing; and
code for determining the sequence of the nucleic acid polymer by assigning basecalls to the peaks.

12. The computer program product of claim 11, wherein the data traces have been preprocessed.

13. The computer program product of claim 12, wherein preprocessing comprises the steps of:
(i) obtaining unprocessed data traces from a plurality of channels of an automated electrophoresis detection apparatus, each channel detecting the products of a DNA sequencing reactions;
(ii) identifying begin and end points in the unprocessed data;
(iii) establishing a baseline in the unprocessed data,
(iv) subtracting the baseline from the unprocessed data to generate the baseline-corrected data; and
(v) separating the baseline-corrected data to generate the data traces, the separating step comprising spectral or leakage separation.

14. The computer program product of claim 11, wherein the electrophoresis detection apparatus uses slab gel electrophoresis; tube gel electrophoresis; or capillary gel electrophoresis.

15. The computer program product of claim 14, wherein the electrophoresis detection apparatus is a MegaBACE capillary sequencing machine.

16. The computer program product of claim 11, further comprising code for generating at least one quality score for at least one basecall.

17. The computer program product of claim 16 wherein the at least one quality score is a gap-quality score, wherein the gap-quality score estimates the probability of a deletion error between two adjacent assigned basecalls.

18. The computer program product of claim 17, wherein the gap-quality score measures degree of noise between the two adjacent assigned basecalls and overly wide peak spacing between the two adjacent assigned basecalls.

19. The computer program product of claim 16, further comprising code for using the quality scores for quality filtering whereby basecalls can be removed or added from the sequence of the nucleic acid polymer during the quality filtering.

20. A computing device comprising a memory device configured to store at least temporarily program instructions for determining a sequence of a nucleic acid polymer, the instructions comprising:
code for obtaining data traces from a plurality of channels of an electrophoresis detection apparatus, each channel detecting the products of a DNA sequencing reactions;
code for combining the data traces by a process comprising the steps of:
(i) applying a cross-correlation coefficient to each of the four data traces to yield four refined traces, wherein the cross-correlation coefficient compares each of the traces with an ideal, Gaussian-shaped peak and wherein the refined traces have narrower peaks than the corresponding data traces;
(ii) combining the four refined traces to yield a composite trace;
code for detecting peaks in the composite trace by a process that is independent of peak spacing; and
code for determining the sequence of the nucleic acid polymer by assigning basecalls to the peaks.

21. The computing device of claim 20, wherein the data traces have been preprocessed.

22. The computing device of claim 21, wherein preprocessing comprises the steps of:
(i) obtaining unprocessed data traces from a plurality of channels of an automated electrophoresis detection apparatus, each channel detecting the products of a DNA sequencing reactions;
(ii) identifying begin and end points in the unprocessed data;
(iii) establishing a baseline in the unprocessed data,
(iv) subtracting the baseline from the unprocessed data to generate the baseline-corrected data; and
(v) separating the baseline-corrected data to generate the data traces, the separating step comprising spectral or leakage separation.

23. The computing device of claim 20, wherein the electrophoresis detection apparatus uses slab gel electrophoresis; tube gel electrophoresis; or capillary gel electrophoresis.

24. The computing device of claim 23, wherein the electrophoreis detection apparatus is a MegaBACE capillary sequencing machine.

25. The computing device of claim 20, further comprising code for generating at least one quality score for at least one basecall.

26. The computing device of claim 25, wherein the at least one quality score is a gap-quality score, wherein the gap-quality score estimates the probability of a deletion error between two adjacent assigned basecalls.

27. The computing device of claim 26, wherein the gap-quality score measures degree of noise between the two adjacent assigned basecalls and overly wide peak spacing between the two adjacent assigned basecalls.

28. The computing device of claim 25, further comprising code for using the quality scores for quality filtering whereby basecalls can be removed or added from the sequence of the nucleic acid polymer during the quality filtering.

29. A method for estimating the probability that a basecall was missed between two adjacent assigned basecalls, the method comprising the steps of:
(a) measuring degree of noise between the two adjacent assigned basecalls;
(b) measuring peak spacing between the two adjacent assigned basecalls; and
(c) computing a gap-quality score, wherein the gap-quality score estimates the probability that a base call was missed between the two adjacent assigned basecalls.

30. The method of claim 29, wherein the basecalls were assigned using the method of claim 1.

31. A computer program product comprising a machine readable medium on which is provided program instructions for estimating the probability that a basecall was missed between two adjacent assigned basecalls, the instructions comprising:
code for measuring degree of noise between the two adjacent assigned basecalls;
code for measuring peak spacing between the two adjacent assigned basecalls; and
code for computing a gap-quality score, wherein the gap-quality score estimates the probability that a base call was missed between the two adjacent assigned basecalls.

32. A computing device comprising a memory device configured to store at least temporarily program instructions estimating the probability that a basecall was missed between two adjacent assigned basecalls, the instructions comprising:
code for measuring degree of noise between the two adjacent assigned basecalls;
code for measuring peak spacing between the two adjacent assigned basecalls; and
code for computing a gap-quality score, wherein the gap-quality score estimates the probability that a base call was missed between the two adjacent assigned basecalls.

33. A method for benchmarking basecaller performance, the method comprising the steps of:
(a) determining a nucleic acid sequence using two base calling algorithms to yield two test sequences;
(b) identifying an aligned sequence between the two test sequences using a sequence comparison algorithm;
(c) comparing the sequence of each of the test sequences with the aligned sequence using the sequence comparison algorithm;
(d) determining high quality left-most and right-most alignments from the comparison;
(e) extending the aligned sequence by identifying a left-most and right-most boundary wherein such boundaries correspond to the left-most and right-most alignments, respectively; and
(f) collecting error statistics over the extended aligned sequence between its left-most and right-most boundaries.

34. The method of claim 33, wherein the sequence comparison algorithm is Blast.

35. The method of claim 33, wherein the error statistics are derived in conjunction with quality scores.

36. The method of claim 35, wherein the quality scores are call quality scores and gap-quality scores.

37. The method of claim 36, wherein preference is given to high call quality scores.

38. The method of claim 36, wherein a low gap-quality indicates a high probability for a deletion error.

39. The method of claim 36, wherein substitution errors are linked to the call quality scores.

40. The method of claim 36, wherein insertion errors are linked to the call quality scores.

41. A computer program product comprising a machine readable medium on which is provided program instructions for benchmarking basecaller performance, the instructions comprising:
code for determining a nucleic acid sequence using two base calling algorithms to yield two test sequences;
code for identifying an aligned sequence between the two test sequences using a sequence comparison algorithm;
code for comparing the sequence of each of the test sequences with the aligned sequence using the sequence comparison algorithm;
code for determining high quality left-most and right-most alignments from the comparison;
code for extending the aligned sequence by identifying a left-most and right-most boundary wherein such boundaries correspond to the left-most and right-most alignments, respectively; and
code for collecting error statistics over the extended aligned sequence between its left-most and right-most boundaries.

42. The computer program product of claim 41, wherein the sequence comparison algorithm is Blast.

43. The computer program product of claim 41, wherein the error statistics are derived in conjunction with quality scores.

44. The computer program product of claim 43, wherein the quality scores are call quality scores and gap-quality scores.

45. The computer program product of claim 44, wherein preference is given to high call quality scores.

46. The computer program product of claim 43, wherein a low gap-quality indicates a high probability for a deletion error.

47. The computer program product of claim 44, wherein substitution errors are linked to the call quality scores.

48. The computer program product of claim 44, wherein insertion errors are linked to the call quality scores.

49. A computing device comprising a memory device configured to store at least temporarily program instructions for benchmnarking basecaller performance, the instructions comprising:
code for determining a nucleic acid sequence using two base calling algorithms to yield two test sequences;
code for identifying an aligned sequence between the two test sequences using a sequence comparison algorithm;
code for comparing the sequence of each of the test sequences with the aligned sequence using the sequence comparison algorithm;
code for determining high quality left-most and right-most alignments from the comparison;
code for extending the aligned sequence by identifying a left-most and right-most boundary wherein such boundaries correspond to the left-most and right-most alignments, respectively; and
code for collecting error statistics over the extended aligned sequence between its left-most and right-most boundaries.

50. The computing device of claim 49, wherein the sequence comparison algorithm is Blast.

51. The computing device of claim 49, wherein the error statistics are derived in conjunction with quality scores.

52. The computing device of claim 51, wherein the quality scores are call quality scores and gap-quality scores.

53. The computing device of claim 52, wherein preference is given to high call quality scores.

54. The computing device of claim 52, wherein a low gap-quality indicates a high probability for a deletion error.

55. The computing device of claim 52, wherein substitution errors are linked to the call quality scores.

56. The computing device of claim 52, wherein insertion errors are linked to the call quality scores.

57. A call quality score, said score being estimated through a process that relies on continuously varying parameters of basecall quality.

58. The call quality score of claim 57, wherein said process does not utilize a look-up table.

59. A gap-quality score, said score estimating the probability that a basecall was missed following a given assigned basecall.

60. The gap-quality score of claim 59, wherein said assigned basecall is determined by the method of claim 1.

61. The gap-quality score of claim 59, wherein said score is derived from the method of claim 29.
